(19) United States
(12) Patent Application Publication
(10) Pub. No.: US 2011/0238018 A1
(43) Pub. Date: Sep. 29, 2011

(54) Intravenous Line Preparation Device

(76) Inventor: Karen McKenzie-Butler, Johnson Creek, WI (US)

(21) Appl. No.: 13/038,186

(22) Filed: Mar. 1, 2011

Related U.S. Application Data
(60) Provisional application No. 61/317,183, filed on Mar. 24, 2010.

(51) Int. Cl. A61M 5/14

(52) U.S. Cl. 604/257

(57) ABSTRACT

The present apparatus is an IV line preparation device including a cap and a chamber cartridge for collecting fluid that needs to be expelled from an IV line before it is inserted into a patient.

### CROSS-REFERENCE TO RELATED APPLICATIONS

This application claims priority to U.S. Provisional Application No. 61/317,183 filed on Mar. 24, 2010.

### FIELD OF INVENTION

The present invention relates to the field of intravenous (IV) line accessories and more particularly to an IV line preparation device.

### BRIEF DESCRIPTION OF THE DRAWINGS

FIG. 1 illustrates an exemplary embodiment of an IV system using tubing cap and collection chamber cartridge system.

FIG. 2a is an exemplary embodiment of an IV line collection chamber cartridge system.

FIG. 2b is an exemplary embodiment of an IV line collection chamber cartridge system in use with an IV tube.

FIG. 2c is an alternative exemplary embodiment of an IV line collection chamber cartridge system

FIG. 3 illustrates an exemplary embodiment of an IV pole belt for use with collection chamber cartridges.

FIG. 4a illustrates the front of an exemplary embodiment of a collection chamber cartridge.

FIG. 4b illustrates an alternative exemplary embodiment of a collection chamber cartridge.

FIG. 4c is a back view of an exemplary embodiment of a collection chamber cartridge.

FIG. 5a illustrates an exemplary embodiment of a tubing cap with inner tubing cap shown in phantom.

FIG. 5b illustrates an exploded view of an exemplary embodiment of a tubing cap, showing tubing, inner tubing cap and outer tubing cap.

FIG. 5c illustrates an exemplary embodiment of a tubing cap in the open position.

FIG. 5d illustrates an exemplary embodiment of a tubing cap in the closed position.

### GLOSSARY

As used herein, the term “intravenous line,” “IV line” or “IV” means any type of tubing or catheter that carries fluid or medication to a human or animal.

As used herein, the term “terminal end” or “distal end” refers to the end of an IV line which contacts an IV line preparation device collection chamber and a patient via a catheter or IV receptacle.

As used herein, the term “surface contour” refers to a contour, shape, indentation or protuberance which is complementary and adapted to allow interlocking, securing or engagement of two components.

As used herein, the term “tubing cap” refers to a component that blocks fluid flow from an IV line.

### BACKGROUND

Intravenous therapy or IV therapy is the giving of liquid substances directly into a vein. The word intravenous simply means “within a vein.” Therapies administered intravenously are often called specialty pharmaceuticals. IV medications are commonly referred to as drips because many systems of fluid administration employ a drip chamber, which prevents air from entering the blood stream (air embolism) and allows an estimate of flow rate.

The intravenous route is the fastest way to deliver fluids and medications throughout the body. Medications, fluids, and transfusions are all delivered intravenously.

A typical intravenous system for a human or veterinary application is comprised of an initiating end with a spike used to access the bag of fluid, medication or blood. The spike is a rigid element with a hollow passageway. Generally, the spike is integrally constructed or connected to IV tubing of any length or diameter. The tubing is a flexible component which tapers slightly and has a rigid member which is open-ended and adapted to terminate at a device which connects to the patient's body through a puncture in a vein (“venipuncture”). In many IV systems, the tapered terminal end containing the rigid member is connected to the patient's venipuncture through a structure known as a Luer lock.

A venipuncture is generally made by an opening in the patient's body created with a needle or other device. An IV is normally inserted into a vein by means of a hollow needle which is then withdrawn to avoid damage to the walls of the punctured vein. The catheter remains attached to the patient and is connected to a source of infusion liquid. The bag of fluid is usually suspended in a bag above the patient to utilize the force of gravity.

Generally, IV tubing must be free of air to avoid air emboli which can be deadly to a patient. An air embolism can develop when the right side of the heart is open to outside air through a disconnected catheter and a negative intrathoracic pressure is present, such as during inspiration.

It has been estimated from animal subject studies with dogs that as little as 20 mL/sec of air can cause an air embolism, and 70 to 150 mL/sec of air can be fatal. Various retrospective clinical studies show that an air embolism due to catheter disconnection has a mortality rate between 29 and 43%. However, any air entering the left side of the heart may enter the cerebral or coronary circulation and result in dangerous air emboli.

In a hospital setting, nurses must clear air from IV tubing by displacing the air in the tubing and allowing the fluid to move through the tubing by the force of gravity to the terminal/end point of the tubing. To ensure all air is purged from the tubing system, nurses usually allow a small amount of fluid to leak out of the terminal/end point of the tubing. Generally, this small amount of fluid is discharged onto an unsanitary receptacle such as a bed, towel, floor, garbage can or sink.

There are also times when a nurse may need to purge or prime the entire IV tubing line. For example, many IV medications and blood are given to patients in a pressurized system. A pump connected to the IV device gently forces the medication or blood into a patient. If the pressure in the IV tubing drops, or the IV tube is partially or fully blocked, a patient's blood may back up into the IV tubing. In order to expel all the blood from the IV tubing, a nurse may have to flush the entire length of the IV tubing, resulting in up to 10 mL of liquid being drained.

IV tubing also contains one or more ports which allow medical professionals to inject medicines into the IV line. Some medications may not be compatible with the liquid being delivered by IV. The solutions will then crystallize in the IV line, forming small solid particles. Not only do the small solid particles impede the flow of liquids through the IV, but it is also undesirable to allow the solid particles to enter the patient's blood stream. In order to remove the solid particulate matter from the IV line, nurses may have to flush and drain the IV tube many times. Up to 20 mL or more may be drained out of the IV tubing to ensure no crystallized particles remain.

Like priming an IV line, nurses often expel liquids drained during flushing the IV tubing into a garbage can or sink, and sometimes directly onto the floor or other non-sterile surface.

Contamination of the tubing during discharge of air and fluid by inadvertently touching the tubing end to a non-sterile surface may require the care provider to discard the entire bag of medication, tubing and other IV apparatus components, resulting in substantial time loss and additional costs to hospitals.

Care providers in high pressure situations may also fail to notice inadvertent contact with a non-sterile surface and therefore fail to maintain sterility in an open tubing system, thus increasing the risk of sepsis (“line sepsis”).

Contamination of IV tubing may also occur when a patient needs to be disconnected from an IV for a period of time. For example, it may be desirable to disconnect a patient from an IV in order for a doctor to perform a thorough examination. Patients also need to shower or bathe. In some instances, examinations and bathing may be timed with IV changes or catheter rotations. However, when a patient needs to be disconnected for a time between IV or catheter changes, the distal end of the IV tubing (the end that connects to the patient's venipuncture) needs to be secured and protected from contamination. In practice, most distal ends of IV tubing are circularly connected to a port further up along the IV tubing. These ports are not sterile and may have bumped against contaminated surfaces.

Alternatively, nurses loop IV tubing around the IV pole or another surface, such as a bed frame or chair arm, in order to keep the tubing out of the way and attempt to prevent contamination of the distal end. These surfaces are not sterile, and looping IV tubing does not prevent the distal end from accidentally being knocked or bumped into other unsterile surfaces. Looping lengths of IV tubing may also pose a safety risk to anyone walking through the area.

IV line related infections are a serious problem for hospitalized patients and pose critical risks to patients in critical care units (CCUs). One of the first signs of an IV line infection is usually fever. Fevers may develop even before any visible sign of an infection at the IV site, resulting in a potential misdiagnosis for patients. Left untreated, a mild IV line infection may develop into a life-threatening situation for patients, particularly patients with already weakened immune systems.

IV line infections not only pose serious medical risks to patients, but also pose an economic burden to the health care system. It is estimated that each bloodstream infection, such as an IV line infection, costs a hospital between $6,000 and $16,000, depending on the type and severity of the infection. At a minimum, hospitals must remove the infected IV, begin a new IV line and treat the infection with antibiotics. As an IV line infection increases in severity, hospitals may also need to consult an infectious disease expert, keep a patient for an extended period to monitor treatment of the infection, and treat any side effects which may result from courses of treatment for the infection, such as kidney damage and sepsis. As of Oct. 1, 2008, Medicare no longer provides reimbursement for hospital-acquired IV infections.

The Joint Commission (TJC), formerly the Joint Commission on Accreditation of Healthcare Organizations (JCAHO) generates national patient safety goals; one goal is eliminate IV infections.

In addition to potential contamination of IV tubing, some medications and fluids may need to be disposed of in a special manner. For example, blood, tissue or other human contaminants need to be disposed of separately from environmentally-unsafe medications. Chemotherapy drugs are also administered by IV and need to be disposed of separately from other drugs and bodily fluids. Proper disposal of medicines, fluids and other contaminated waste prevents patients and medical staff from coming in contact with potentially hazardous material and toxins from being released into the environment. By draining IV liquids, medicines or other solutions into general receptacles, such as trash cans or sinks, or onto unspecified and often easily accessible surfaces, such as floors and bedding, nurses are exposing patients, colleagues and others to potentially hazardous materials.

It is desirable to have a closed IV system that avoids the risk of air-flow caused embolisms.

It is further desirable to have a method of discharging an IV line that has backed up with blood.

It is further desirable to have an IV system which does not require a care provider to search for a safe receptacle in which to discharge air and fluid from an IV line in high stress situations and emergencies.

It is further desirable to have an IV system which reduces the risk of IV line contamination when bleeding the IV line system.

It is further desirable to have a disposable and safe receptacle in which to discharge air and fluid from an IV line that allows for proper disposal of the discharged fluids.

### SUMMARY OF THE INVENTION

The present invention is an intravenous line preparation system comprised of a collection chamber which is removably and securely attached to an IV pole. The collection chamber contains a housing surrounding a sterile internal chamber with an aperture adapted to receive an intravenous line. The system also includes an IV priming cap consisting of an inner cap with a single aperture and an outer cap with a solid bottom and a plurality of apertures on the sides of the outer cap. Inner and outer cap are adapted to slidingly engage one another so that when outer cap is slid away from inner cap, the single aperture on said inner cap is exposed and fluid is free to flow from the intravenous line through the single aperture in inner cap and out the plurality of apertures in outer cap. When outer cap is slid against inner cap, the single aperture is physically sealed against the outer cap, and flow from the intravenous line is stopped.

### DETAILED DESCRIPTION OF INVENTION

For the purpose of promoting an understanding of the present invention, references are made in the text to exemplary embodiments of an intravenous line preparation device, only some of which are described herein. It should be understood that no limitations on the scope of the invention are intended by describing these exemplary embodiments. One of ordinary skill in the art will readily appreciate that alternate but functionally equivalent designs, materials and components may be used. The inclusion of additional elements may be deemed readily apparent and obvious to one of ordinary skill in the art. Specific elements disclosed herein are not to be interpreted as limiting, but rather as a basis for the claims and as a representative basis for teaching one of ordinary skill in the art to employ the present invention.

It should be understood that the drawings are not necessarily to scale; instead, emphasis has been placed upon illustrating the principles of the invention. In addition, in the embodiments depicted herein, like reference numerals in the various drawings refer to identical or near identical structural elements.

Moreover, the terms “substantially” or “approximately” as used herein may be applied to modify any quantitative representation that could permissibly vary without resulting in a change in the basic function to which it is related.

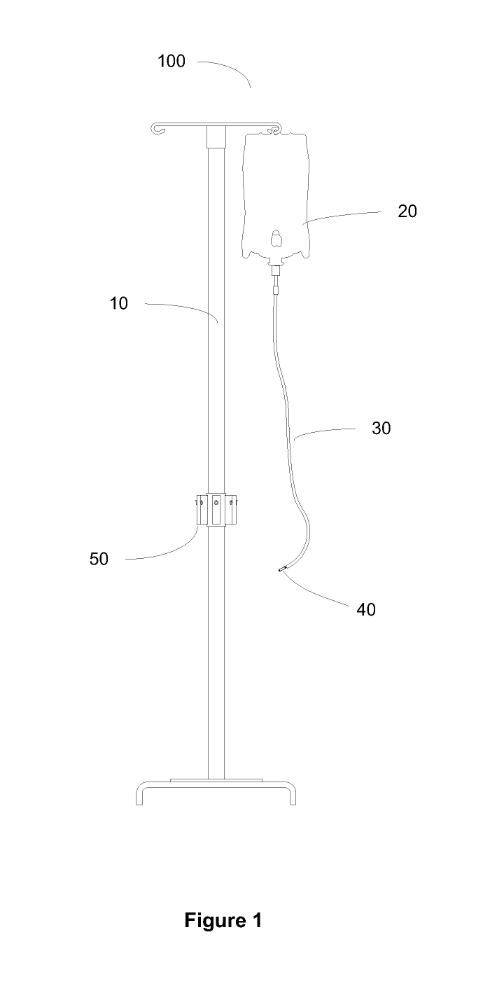

FIG. 1 illustrates an exemplary embodiment of IV system 100 using tubing cap 40 and collection chamber cartridge system 50. IV pole 10 supports IV bag 20, which contains a fluid such as a liquid solution, blood or medication. In the exemplary embodiment shown, IV tubing 30 runs from IV bag 20 to collection chamber cartridge system 50 for priming. Tubing cap 40 is secured to the distal end of IV tubing 30 and allows IV tubing 30 to be primed within sterile internal chambers 52 (not shown), reducing the risk for contamination.

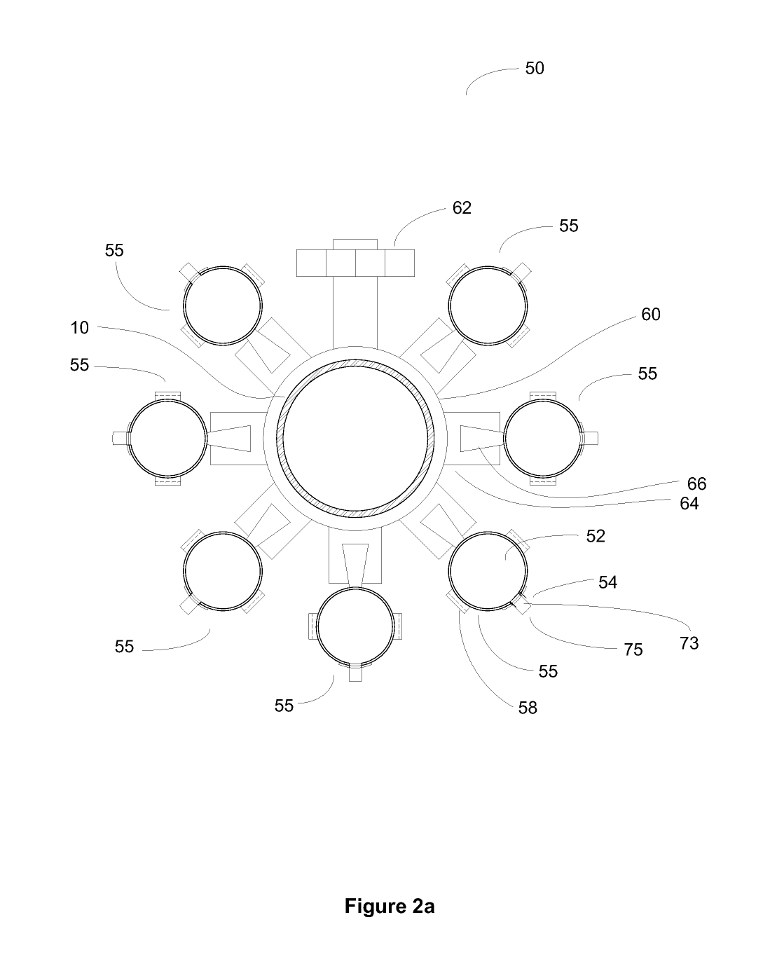

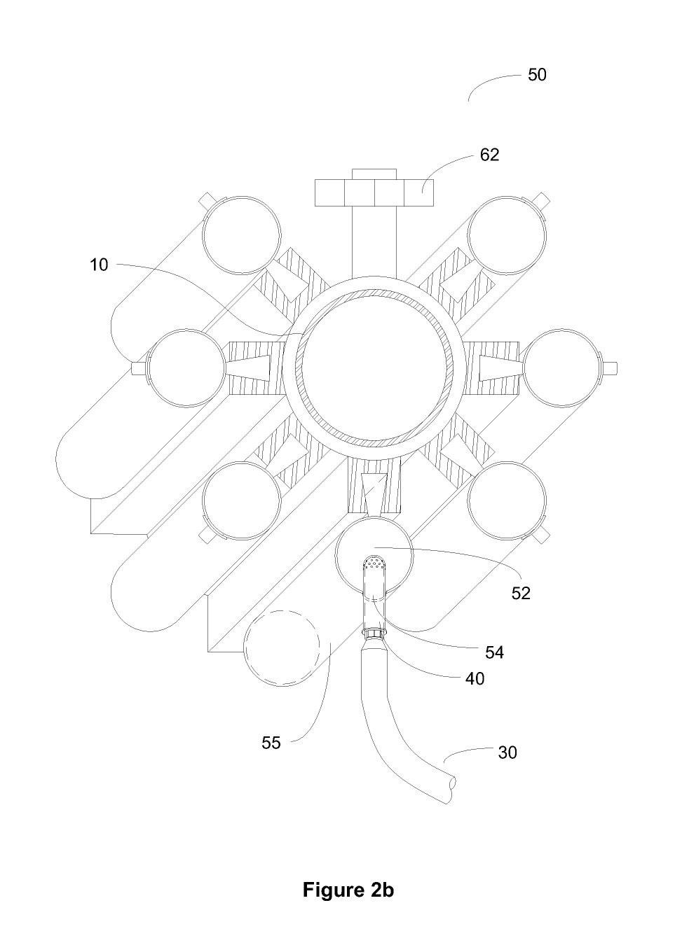

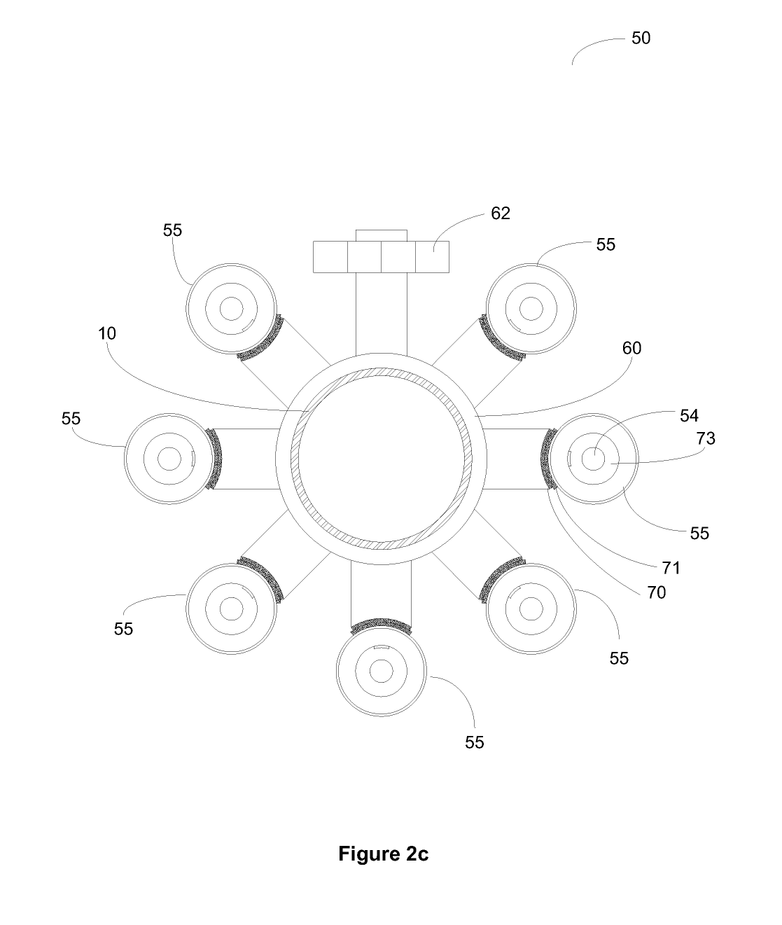

FIG. 2a illustrates an exemplary embodiment of IV line collection chamber cartridge system 50. In the exemplary embodiment shown in FIG. 2a, collection chamber cartridge system 50 contains a plurality of chamber cartridges 55, each with aperture 54 and sterile internal chamber 52. As shown in FIG. 2a, each aperture 54 opens to sterile internal chamber 52, which is protected from the external environment by covering component 73, which in the exemplary embodiment shown is a pull tab.

In further exemplary embodiments, covering components 73 may be any structure or device known in the art to cover apertures 54 and protect them from the external environment, including, but not limited to, seals, surfaces which are punctured, covers, caps, plugs and combinations thereof.

In the exemplary embodiment shown, chamber cartridges 55 are secured to IV pole 10 using belt 60. Chamber cartridges 55 contain protuberances 64, with belt 60 containing corresponding tracks 66. Chamber cartridges 55 may be securely slid onto and off of belt 60 using gripping members 58, which are shown as small contoured protuberances from the sides of chamber cartridges 55. Gripping members 58 allow a user to easily grasp chamber cartridges 55. In further exemplary embodiments, gripping members 58 may further contain a texture or coating to help a user grip chamber cartridges 55. In still further exemplary embodiments, gripping members 58 may be any other structure known in the art to help a user grip chamber cartridges 55, including, but not limited to, loops, handles, bars, tabs, or combinations of these structures. In yet further exemplary embodiments, chamber cartridges 55 may contain more or fewer gripping members 58, and gripping members 58 may be positioned differently on chamber cartridges 55.

As shown in FIG. 2a, tracks 66 extend vertically parallel with IV pole 10 to create a sliding track for chamber cartridges 55 to be quickly and easily attached and removed. In further exemplary embodiments, belt 60 may be omitted, and tracks 66 may be directly attached to IV pole 10. In still further exemplary embodiments, chamber cartridges 55 may be removably secured directly to IV pole 10. In the exemplary embodiment shown, chamber cartridges 55 are designed to be individually disposable in order to quickly and safely dispose of waste products. In further exemplary embodiments, chamber cartridges 55 may be permanently affixed to belt 60 or IV pole 10.

In the exemplary embodiment shown in FIG. 2a, belt 60 is adapted to secure seven chamber cartridges 55. In further exemplary embodiments, belt 60 may be adapted to secure more or fewer chamber cartridges. Belt 60 may also be positioned higher or lower on IV pole to accommodate users or lengths of IV tubing 30 (not shown).

As shown in FIG. 2a, tracks 66 protrude slightly from belt 60 in order to hold chamber cartridges 55 a distance from IV pole 10. Chamber cartridges 55 may be easier to connect and disconnect from belt 60 if not immediately adjacent with IV pole 10. Keeping chamber cartridges 55 a distance from IV pole 10 also makes them more easily accessible if a nurse would need to prime or flush an IV in an emergency situation.

Also visible on belt 60 is securing member 62, which in the exemplary embodiment shown is a tightening member which tightens belt 60 around IV pole 10 and secures it in place. In the exemplary embodiment shown in FIG. 2a, belt 60 is a loop made of a semi-flexible material which is slid onto IV pole 10 and tightened around IV pole 10 using securing member 62. In further exemplary embodiments, belt 60 may be hinged or contain two loose ends in order to fit around IV pole 10. In still further exemplary embodiments, securing member 62 may be different structures or materials known in the art to removably secure belt 60 to IV pole 10, such as elastic, clips, braces, clasps, ties, buttons, snaps, hook-and-loop fasteners, hook-and-eye fasteners, magnetic materials, or any other structure or material known in the art to securely and removably attach belt 60 to IV pole 10.

FIG. 2b is an exemplary embodiment of IV line collection chamber cartridge system 50 in use with IV tube 30 and IV tubing cap 40. Covering component 73 (not shown) is removed from chamber cartridge 55. IV tubing cap 40 connected to IV tube 30 projects through exposed aperture 54 into sterile internal chamber 52. When IV tube 30 is primed or flushed, fluid is contained within sterile internal chamber 52, and IV tubing cap 40 is not exposed to unsanitary surfaces, such as garbage cans, floors, sinks or bedding.

In the exemplary embodiments shown in FIGS. 2a and 2b, apertures 54 are located on the front surface of chamber cartridges 55. A greater length of IV tubing needs to be used to reach an aperture located on the front or side surfaces of chamber cartridges 55, which prevents tangles or other flow impedences when priming or flushing an IV line. Positioning apertures 54 near the top of chamber cartridge 55 increases the amount of usable internal volume as well. However, in further exemplary embodiments, apertures 54 may be located on any surface of chamber cartridge 55 as long as there is a usable internal volume for liquid expelled from an IV tube during priming or flushing.

FIG. 2c is an alternative exemplary embodiment of IV line collection chamber cartridge system 50. Belt 60 is secured around IV pole 10 and contains hook-and-loop portions 70. Chamber cartridges 55 contain corresponding hook-and-loop portions 71 to removably and securely attach chamber cartridges 55 to belt 60.

Chamber cartridges 55 contain apertures 54, which are covered by covering components 73. In the exemplary embodiment shown in FIG. 2c, apertures 54 are shown on the top of chamber cartridges 55. Positioning apertures 54 on the tops of chamber cartridges 55 maximizes the effect of gravity when priming or flushing IV tubes. However, less IV tubing length is used to reach apertures 54 when located on the tops of chamber cartridges 55, and belt 60 may need to be positioned lower on IV pole 10 to prevent kinks in IV tube 30 (not shown) or loops which may hinder flow.

In the exemplary embodiments described in FIGS. 2a, 2b and 2c, belt 60 uses tracks 66 with protuberances 64 or hook-and-loop portions 70, 71 to secure chamber cartridges 55 to belt 60. In further exemplary embodiments, belt 60 may contain other structures known in the art to allow chamber cartridges 55 to be securely and removably attached to IV pole 10. For example, belt 60 may contain hook-and-eye fasteners, buttons, snaps, clamps, clips, braces, contours, ties, or any combination of these structures. In further exemplary embodiments, belt 60 may be configured to hold more or fewer chamber cartridges or chamber cartridges of varying size.

While in the exemplary embodiments described in FIGS. 2a, 2b and 2c, belt 60 is made from a semi-flexible material adapted to be secured around IV pole 10 using securing member 62, in further exemplary embodiments, belt 60 may be made of an elastic material adapted to secure to IV pole 10 by compression. In yet further exemplary embodiments, belt 60 may be of any material known in the art for providing a surface for securely and removably attaching chamber cartridges 55. Belt 60 may also be made of a rigid material manufactured to conform to the shape of IV pole 10 or a flexible material which may be physically adapted to conform and attach to IV pole 10.

In the exemplary embodiment shown in FIGS. 2a, 2b and 2c, securing member 62 is shown as a tightening component which tightens belt 60 tight around IV pole 10. Belt 60 may also use a different securing member 62 to attach to IV pole 10, such as elastic, compression force, clips, clasps, adhesive, braces, contours, or any other device or structure known in the art to secure belt 60 to IV pole 10 or combination of such structures or devices.

In still further exemplary embodiments, belt 60 may be omitted entirely and tracks 66, hook-and-loop portions 70 or any other attachment means affixed directly to IV pole 10. Tracks 66 or other attachment means may also be manufactured as an integral component with IV pole 10.

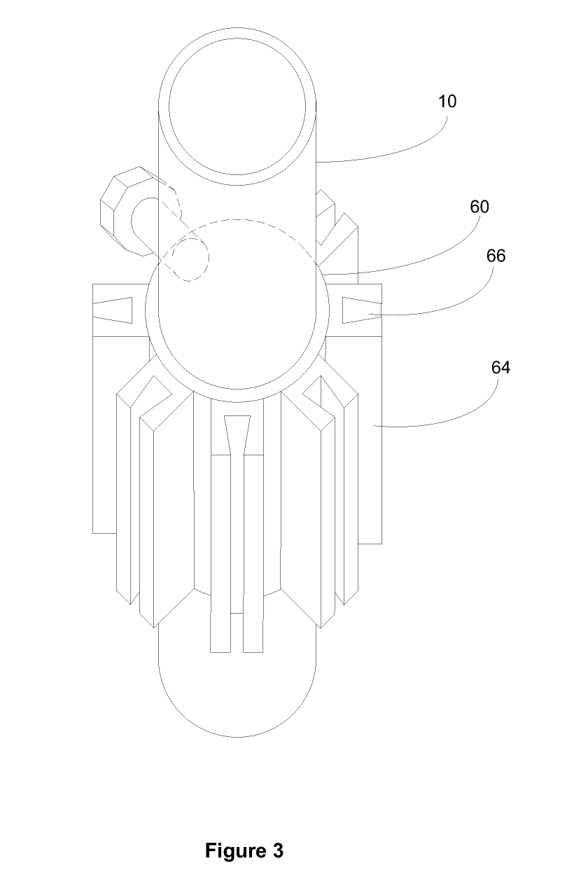

FIG. 3 is an exemplary embodiment of belt 60 without chamber cartridges 55 (not shown). Tracks 66 are shown running parallel with IV pole 10 for the length of belt 60. As shown in FIG. 3, tracks 66 are triangular shaped grooves in protruding surfaces extending perpendicularly from belt 60. In further exemplary embodiments, tracks 66 may be differently configured or be longer, shorter, narrower or wider to correspond to a differing structure on chamber cartridges 55 (not shown). In some exemplary embodiments, tracks 66 may include a stop to prevent chamber cartridge 55 from progressing down track 66.

In the exemplary embodiment shown in FIG. 3, one track 66 corresponds to one chamber cartridge 55 (not shown). In further exemplary embodiments, tracks may be configured so that each chamber cartridge 55 (not shown) is secured using two or more tracks. In still further exemplary embodiments, belt 60 may contain protuberances 64 (not shown) and chamber cartridges 55 (not shown) may contain tracks 66.

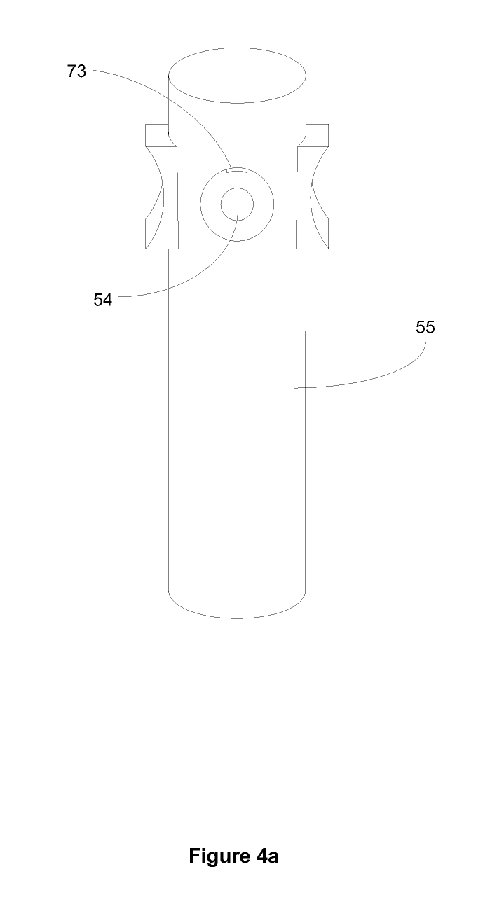

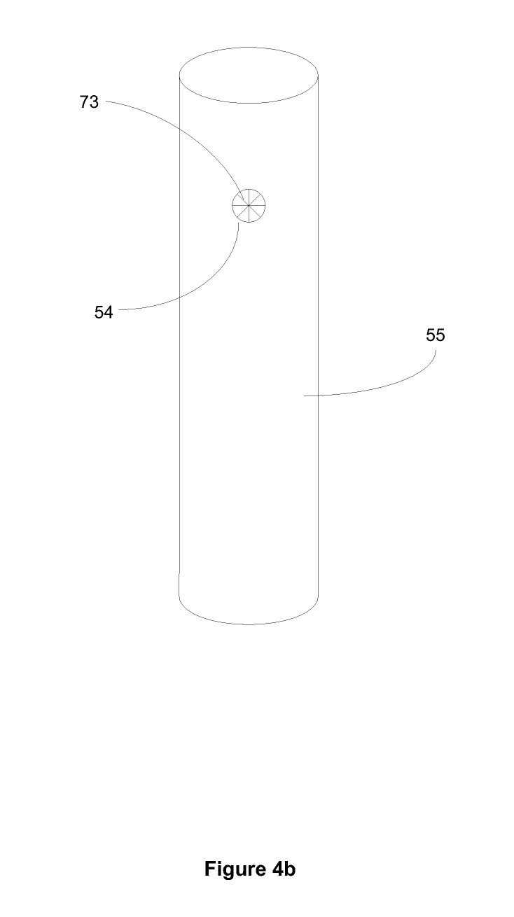

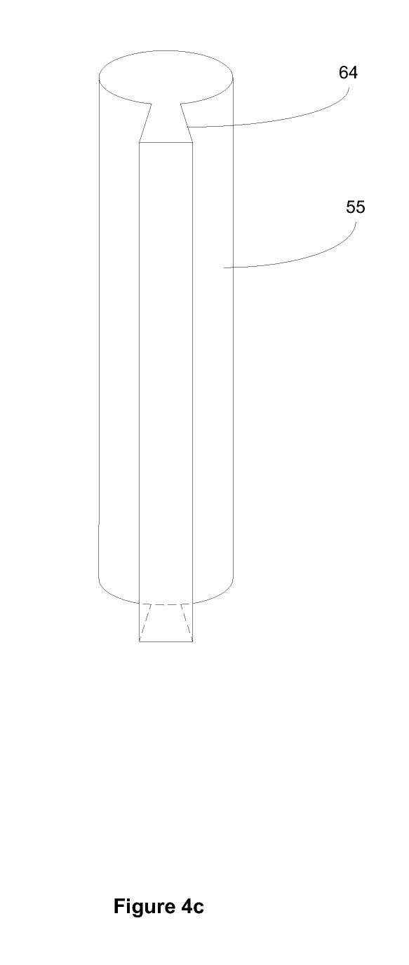

FIG. 4a is an exemplary embodiment of chamber cartridge 55. Chamber cartridge 55 is a hollow oblong cylinder with sterile internal chamber 52 (not shown) having a volume of approximately 10 milliliters. 10 milliliters is the approximate volume contained by an IV line. In the exemplary embodiment shown in FIG. 4a, an IV line may be primed or completely flushed. In further exemplary embodiments, sterile internal chamber 52 (not shown) may have a larger volume, such as 20 milliliters or more, to accommodate multiple complete flushes if an IV line, if necessary. Sterile internal chamber 52 (not shown) may also have a smaller volume for use when only priming an IV line or securing an IV line when it is not in use.

In further exemplary embodiments, chamber cartridge 55 may be a different shape, such as squared or contoured. In some exemplary embodiments, chamber cartridge 55 may contain a bulbous bottom portion to accommodate larger internal volumes. In still further exemplary embodiments, chamber cartridge 55 may contain a narrowed portion to contain aperture 54 and receive IV tube 30 (not shown) with tubing cap 40 (not shown).

As shown in FIG. 4a, chamber cartridge 55 is a single component with aperture 54 opening directly into sterile internal chamber 52 (not shown) and gripping members 58. In further exemplary embodiments, chamber cartridge 55 may be two or more distinct components. For example, chamber cartridge 55 may contain one component which acts as sterile internal chamber 52 (not shown) and a separate component adapted to receive IV tube 30 (not shown) with tubing cap 40 (not shown).

Chamber cartridge 55 also contains covering component 73 which seals sterile internal chamber 52 (not shown) from the environment. In the exemplary embodiment shown, covering component 73 is a pull-off tab. In further exemplary embodiments, covering component 73 may be any other structure or device known in the art to cover aperture 54 and prevent sterile internal chamber 52 (not shown) from being exposed to the external environment.

FIG. 4b is an exemplary embodiment of an alternative chamber cartridge 55. In the exemplary embodiments shown, covering component 73 is a rubber seal with spoke-like serrations. Covering component 73 allows IV tube 30 (not shown) with tubing cap 40 (not shown) to penetrate into sterile internal chamber 52 (not shown) when tubing cap 40 (not shown) pushes in the center of covering component 73. Tubing cap 40 (not shown) breaks the serrations and allows tubing cap 40 to penetrate into sterile internal chamber 52 (not shown). In some exemplary embodiments, aperture 54 may also contain protuberance 75 (not shown) which extends into sterile internal chamber 52 (not shown) from aperture to provide a securing platform for IV tube 30 (not shown) with tubing cap 40 (not shown). Together, covering component 73 and protuberance 75 (not shown) help stabilize IV tube 30 (not shown) with tubing cap 40 (not shown) in chamber cartridge 55 during priming and flushing or if distal end of IV tube 30 (not shown) needs to disconnected from a patient for a period of time.

FIG. 4c is a back view of an exemplary embodiment of chamber cartridge 55. Chamber cartridge 55 contains protuberance 64, which runs the length of chamber cartridge 55 parallel with IV pole 10 (not shown). As shown in FIG. 4c, protuberance 64 is a triangular projection from chamber cartridge 55 which corresponds to track 66 (not shown) in belt 60 (not shown). In further exemplary embodiments, protuberance 64 may be of a different shape or configuration, and more than one protuberance 64 may correspond to a track 66 (not shown). In further exemplary embodiments, chamber cartridge 55 may be secured to belt 60 (not shown) using any structure or device known in the art to removably secure chamber cartridge 55 to belt 60, including, but not limited to, elastic, clips, braces, clasps, ties, buttons, snaps, hook-and-loop fasteners, hook-and-eye fasteners, magnetic materials, or any other structure or device, or combination thereof, able to removably secure chamber cartridge 55 to belt 60. Protuberances 64 may also contain a stop to prevent chamber cartridge 55 from progressing further down tracks 66 (not shown).

Chamber cartridges 55 may be manufactured out of any material known in the art to contain medical wastes, including, but not limited to, plastics and unreactive materials. In some exemplary embodiments, chamber cartridges 55 may be color-coded to correspond to disposal procedures. For example, chamber cartridges 55 for disposing of radioactive wastes may be a different color than chamber cartridges 55 for disposing of blood wastes. Other colors may be used to designate waste that does not need pre-disposal treatment, waste that does need pre-disposal treatment, or other designations as specified in hospital guidelines.

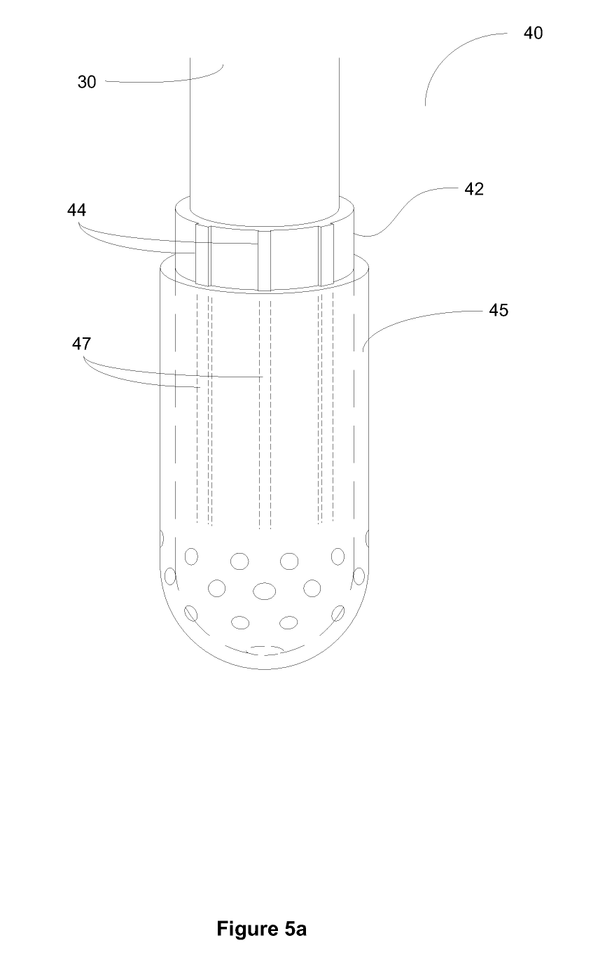

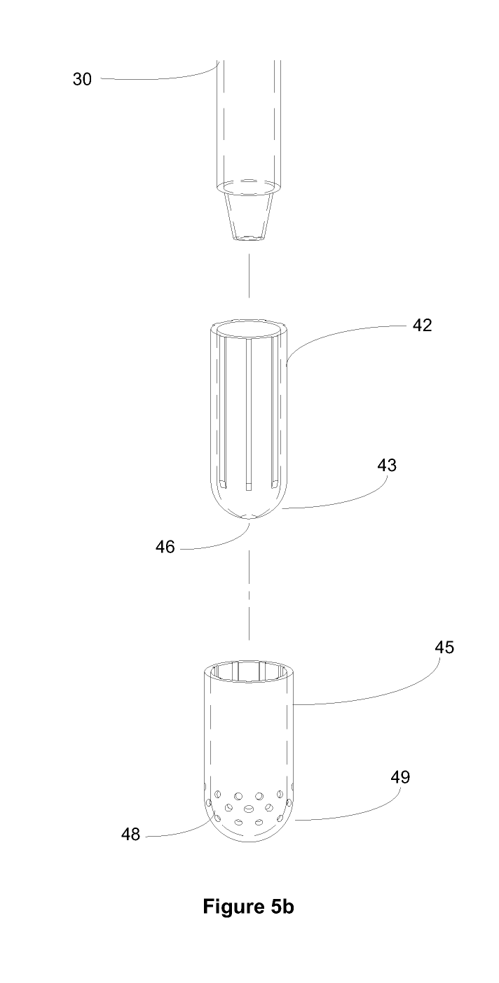

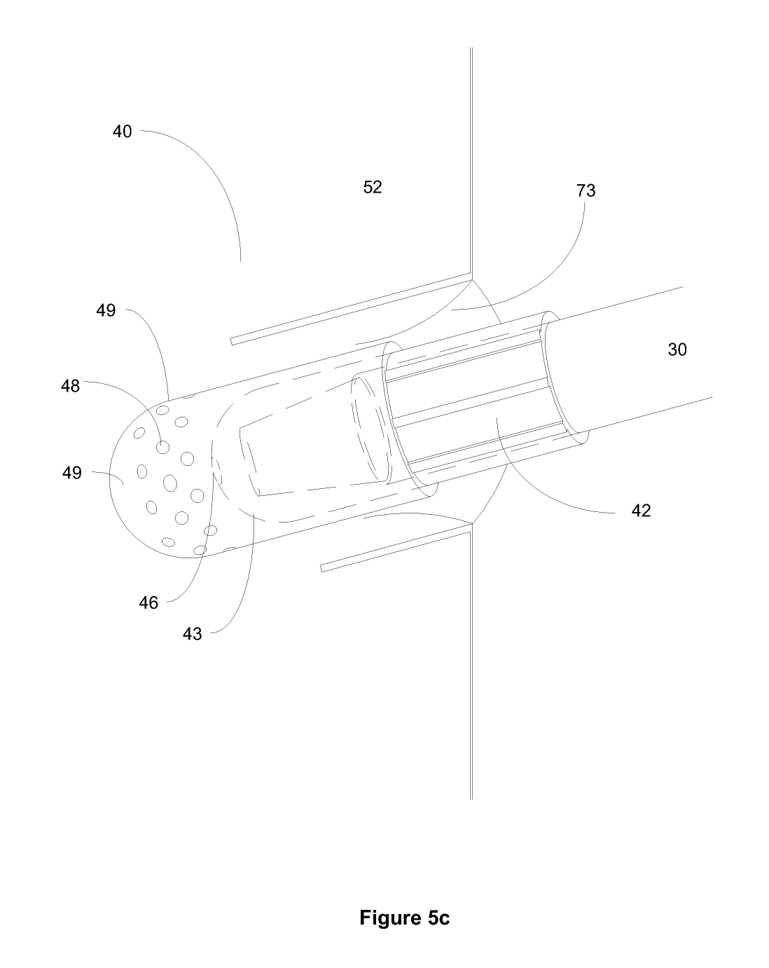

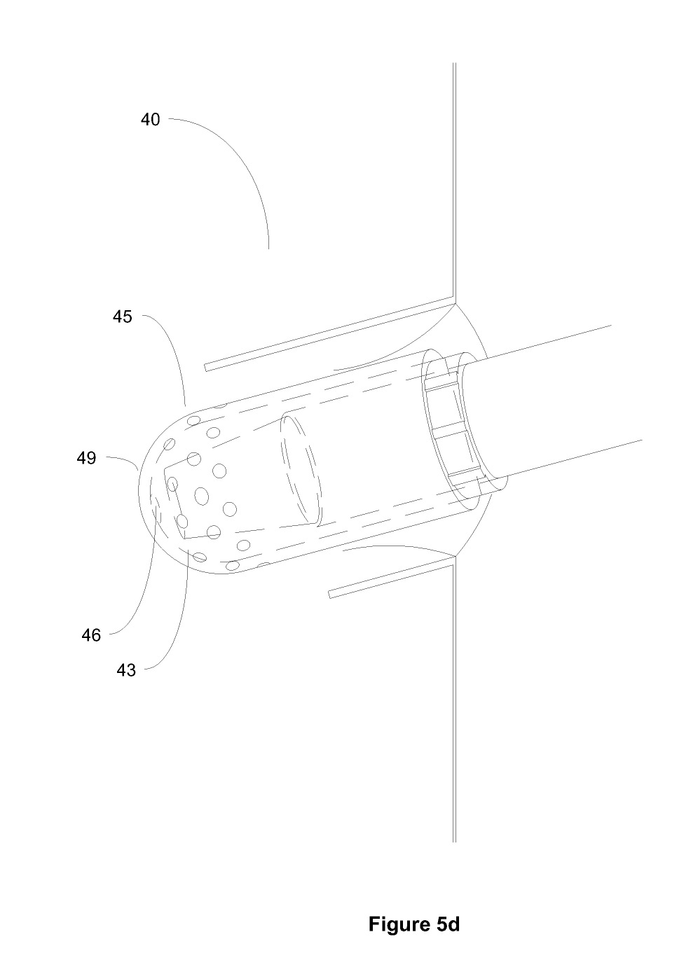

FIG. 5a illustrates a side view of an exemplary embodiment of tubing cap 40. In the embodiment shown, tubing cap 40 is comprised of inner cap 42 (shown in phantom) and outer cap 45. Inner cap 42 is an open-ended tubular structure which surrounds the terminal end of IV tubing 30. Inner cap 42 further includes inner cap surface contour 44 and outer cap 45 includes a complementary outer cap surface contour 47.

In the embodiment shown, inner cap surface contour 44 is a series of protuberances and outer cap surface contour 47 is a series of complementary apertures adapted to receive inner cap surface contour 44. In other embodiments, inner cap surface contour 44 and outer cap surface contour 47 may include snaps, flanges, brackets, hinges, rings, or combinations thereof.

In further exemplary embodiments, tubing cap 40 may be a barrier, a stopper, a sterile seal, a membrane, a gage, a stopcock, a plug, a twisting component, or a breakable structure and may be retractable or non-retractable.

FIG. 5b illustrates an exploded view of an exemplary embodiment of IV tube 30, inner cap 42 and outer cap 45. In the embodiment shown, inner cap tip 43 has opening 46 and outer cap tip 49 has a plurality of apertures 48. In other embodiments, there may be more or fewer apertures or apertures of varying shapes and sizes.

FIG. 5c illustrates a side view of an exemplary embodiment of tubing cap 40 in the open position. Tubing cap 40 is in the open position when inner cap 42 is not pushed against outer cap tip 49. When tubing cap 40 is in the open position, fluid is allowed to flow freely from tubing 30 through opening 46 in inner cap tip 44 into outer cap 45 and out apertures 48 in outer cap tip 49.

In the exemplary embodiment shown, tubing cap 40 can be advanced to the closed position by inserting inner cap 42 further into outer cap 45 so that inner cap tip 43 pushes against outer cap tip 49. To open tubing cap 40, inner cap 42 is retracted from outer cap 45.

Also shown in FIG. 5c are protuberances 75 which extend from aperture 54 into sterile internal chamber 52 from aperture to provide a securing platform for IV tube 30 with tubing cap 40. Protuberance 75 helps stabilize IV tube 30 with tubing cap 40 in chamber cartridges 55 during priming and flushing of IV line 30 or if distal end of IV tube 30 needs to disconnected from a patient for a period of time. In the exemplary embodiment shown, protuberances 75 extend from the top and bottom of aperture 54, but in further exemplary embodiments may by a continuous or non-continuous lip extending inward around aperture 54 or extend from the sides of aperture 54.

As shown in FIG. 5c, protuberance 75 extends from aperture 54 at a slight downward angle into sterile internal chamber 52. A downward angle may allow gravity to help liquid drain from IV tube 30. In further exemplary embodiments, protuberances 75 may be perpendicular to chamber cartridge 55. In still further exemplary embodiments, protuberances 75 may angle slightly upward or horizontally relative to the sides of chamber cartridges 55 to help secure IV tube 30 with tubing cap 40 within apertures 54.

In still further exemplary embodiments, protuberances 75 may contain a textured or coated surface to help engage and secure IV tube 30 with tubing cap 40. For example, protuberances 75 may contain a friction-increasing texture or coating, or contain contours which engage corresponding contours on tubing cap 40.

As illustrated in FIG. 5c, aperture 54 contains punctured covering component 73. As tubing cap 40 pushed against covering component 73, the seal created by covering component 73 was broke, and tubing cap 40 enters sterile internal chamber 52. Once tubing cap 40 is removed from chamber cartridge 55, sterile internal chamber 52 is no longer separated from the external environment.

FIG. 5d illustrates a side view of an exemplary embodiment of tubing cap 40 in the closed position. When tubing cap 40 is in a closed position, i.e., when inner cap tip 43 is pushed up against outer cap tip 49, the inner surface of outer cap tip 49 acts as a stop and blocks fluid flow from opening 46 in inner cap tip 43.

The inner surface of outer cap tip 49 may be comprised of the same material as outer cap 45 or a different material that facilitates a better seal (e.g., softer plastic, foam, fabric, rubber, fiber, nylon, cotton or equivalents). In various embodiments, the inner surface of outer cap tip 49 could be flattened, more rounded or adapted to receive the terminal end of tubing 30 in a manner which forms a seal when tubing 30 abuts against the inner surface of outer cap tip 49.

In the exemplary embodiments shown in FIGS. 5a-5d, tubing cap 40 is shown as two components. In further exemplary embodiments, tubing cap 40 may be manufactured to be a single component or structure. In still further exemplary embodiments, tubing cap 40 may be an integral component of aperture 54. In yet further exemplary embodiments, inner cap 44 and outer cap 45 may be manufactured as separate components but integrally assembled so that inner cap 44 and outer cap 45 may not be disassembled.

What is claimed is:

1. An intravenous line preparation device comprised of:
at least one collection chamber component having a housing surrounding an inner chamber, said housing further including at least one aperture adapted to receive an intravenous line; and
at least one securing structure removably securing said at least one collection chamber component to an IV pole.

2. The device of claim 1 wherein said securing structure releasably engages said IV pole.

3. The device of claim 1 wherein said at least one securing structure is permanently affixed to said IV pole.

4. The device of claim 1 wherein said at least one securing structure removably engages said IV pole through an engaging component.

5. The device of claim 1 which includes seven collection chamber components.

6. The device of claim 1 wherein said at least one collection chamber is removably connected to said engaging component.

7. The device of claim 1 which further includes a priming cap adapted to engage said intravenous line and interface with said aperture.

8. The device of claim 7 wherein said priming cap is comprised of
an inner cap having a single aperture on the tip of said inner cap and at least one surface contour, and
an outer cap with a plurality of apertures on the sides of said outer cap and at least one aperture corresponding to said at least one surface contour on said inner cap,
wherein said outer cap slidingly engages said inner cap.

9. An intravenous line preparation system comprised of:
at least one collection chamber component having a housing surrounding an inner chamber, said housing further including at least one aperture adapted to receive an intravenous line;
an engaging component adapted to securely engage an IV pole, said engaging component containing at least one securing structure adapted to removably secure said at least one collection chamber component to said engaging component; and
a priming cap adapted to engage said intravenous line and interface with said at least one aperture.

10. The system of claim 9 wherein said priming cap is comprised of
an inner cap having a single aperture on the tip of said inner cap and at least one surface contour, and
an outer cap with a solid bottom surface, a plurality of apertures on the sides of said outer cap and at least one aperture corresponding to said at least one surface contour on said inner cap,
wherein said outer cap slidingly engages said inner cap.

11. The system of claim 10 wherein said single aperture on said inner cap physically contacts said solid bottom surface of said outer cap to prevent liquid from leaving said inner cap.

12. The system of claim 10 wherein said outer cap slidingly exposes said single aperture on said inner cap to allow said intravenous line to drain through said plurality of apertures in said outer cap.

13. The system of claim 9 wherein said at least one securing structure comprises
at least one protuberance securely attached to said housing, and
at least one aperture corresponding to said at least one protuberance securely attached to said engaging component.

14. The system of claim 9 wherein said at least one securing structure is selected from the group consisting of hook-and-loop fasteners, hook-and-eye fasteners, sliding track structures, buttons, snaps, clamps, clips, braces, contours, ties, or combination thereof.

15. The system of claim 9 wherein said at least one aperture is covered by a covering component.

16. The system of claim 9 wherein said housing further includes a protuberance extending from said at least one aperture into said inner chamber.

17. The system of claim 9 wherein said housing further includes at least one gripping member selected from the group consisting of loops, handles, bars, tabs, contours, textured surfaces or combinations thereof.

18. The system of claim 9 wherein said aperture is positioned on the side of said housing.

19. The system of claim 9 wherein said inner chamber has a volume of at least 10 milliliters.

20. The system of claim 9 wherein said engaging component is adapted to secure more than one collection chamber component to said IV pole.
